(19) United States
(12) Patent Application Publication
(10) Pub. No.: US 2016/0331933 A1
(43) Pub. Date: Nov. 17, 2016

(54) DUAL DEFLECTION PULL WIRE RING

(71) Applicant: Medtronic CryoCath LP, Toronto (CA)

(72) Inventor: Kevin A. KNUTSEN, Ramsey, MN (US)

(21) Appl. No.: 14/712,490

(22) Filed: May 14, 2015

(51) Int. Cl. A61M 25/01; A61B 5/00; A61B 18/14

(52) U.S. Cl. CPC A61M 25/0147; A61B 18/1492; A61B 5/6852; A61M 2025/015; A61B 17/00234

(57) ABSTRACT

A system and method for anchoring a pull wire within the distal portion of a device elongate body. A steering deflection mechanism includes a pull ring having a plurality of apertures and two receiving slots. For example, the pull ring may include a first aperture and a second aperture diametrically opposed to each other and a first receiving slot and a second receiving slot diametrically opposed to each other. Each receiving slot may be defined by the band and extend within the band from a first edge of the band to a distance from a second edge of the band. This configuration may enable dual deflection of the distal portion of the device elongate body.

### STATEMENT REGARDING FEDERALLY SPONSORED RESEARCH OR DEVELOPMENT

n/a

### CROSS-REFERENCE TO RELATED APPLICATION

n/a

### FIELD OF THE INVENTION

The present invention relates to a device, system, and method for anchoring a catheter pull wire within the distal end of a catheter and providing for dual deflection.

### BACKGROUND OF THE INVENTION

Catheters are commonly used to perform medical procedures within very small spaces in a patient's body, and most procedures mandate precise catheter navigation. A catheter used to perform many ablation and mapping procedures generally includes a handle and a flexible elongate body having a distal end. Steering the distal end of a catheter can be difficult, especially as the elongate body passes through a tortuous vascular path.

Catheter tip steering is often accomplished with the use of one or more pull wires attached to a pull ring within the distal end of the catheter shaft at one end, and coupled to a steering control mechanism housed within the handle at the other end. Manipulation of the steering control mechanism will deflect the catheter tip through pulling or releasing pull force pressure on the one or more pull wires. The pull force exerted on a pull wire within a standard-sized catheter is often quite large, for example, in excess of ten pounds, and the pull force required is increased for thicker or longer catheters. Therefore, the point of connection between a pull wire and the pull ring must be able to withstand this force in order to preserve the integrity of the steering system.

Currently, the most frequently used type of joining method between a pull wire and pull ring is welding the distal end of a flat pull wire to an outer surface of a pull ring. However, this joining method is very susceptible to stress fractures and peeling as a pull force is exerted repeatedly over time (referred to herein as “destructive pull force” to distinguish from the pull force necessary to steer the catheter tip). Sometimes a round pull wire may be used that has a flattened distal end for attachment to the pull ring, but this does not provide a significant benefit over using a flat pull wire. The point in the pull wire at which the flat distal end transitions into the rounded wire body becomes another stress point when the catheter tip is repeatedly deflected. As a result, the pull wire may become detached from the pull ring, or may break at the stress point. Further, it is not uncommon for pull rings to become detached from the inside of the catheter shaft and creep away from the distal end of the catheter as a result of repeated deflections and manipulations.

A system and method of anchoring a pull wire to a pull ring in a catheter deflection mechanism is provided herein. Further provided is a system and method to enhance the strength of the join between a pull wire and pull ring so that the mechanism may reach higher levels of pull force while maintaining design integrity. For example, the system and method include an increased amount of weld sites for attaching a pull wire to a pull ring.

### SUMMARY OF THE INVENTION

The present invention advantageously provides a device, system, and method for anchoring a catheter pull wire within the distal end of a catheter and providing for dual deflection. A catheter steering assembly may include a first pull wire and a second pull wire and a continuous annular band including therein a first aperture, a second aperture, and a first receiving slot, and a second receiving slot, the first aperture being approximately 180° from the second aperture and the first receiving slot being approximately 180° from the second receiving slot, and at least a portion of the first pull wire being received within the first aperture and at least a portion of the second pull wire being received within the second aperture. For example, each of the first and second pull wires may each have a circular cross section and each of the first and second receiving slots may have a circular cross section that is sized to longitudinally receive a portion of one of the first pull wire and the second pull wire. Alternatively, the first and second pull wires each may have a rectangular cross section and each of the first and second receiving slots may have a rectangular cross section that is sized to longitudinally receive a portion of one of the first pull wire and the second pull wire. Further, the first and second apertures each may be equidistant from the first edge and second edge. The band may include a thickness, a first edge, a second edge, and a height between the first edge and second edge, the height being greater than the thickness. The first and second receiving slots may each extend within the band from the first edge to a distance from the second edge. For example, the distance from the second edge is between approximately 10% to approximately 50% of the entire height of the band. The band may further include a fusible band, which may be composed of a thermoplastic, in contact with an inner surface of the band.

A catheter steering assembly may include a first pull wire having a distal end and a second pull wire having a distal end, the first and second pull wires each being flat in cross section and a continuous annular band including therein a first aperture, a second aperture, a first receiving slot and a second receiving slot, the first aperture being approximately 180° from the second aperture and the first receiving slot being approximately 180° from the second receiving slot, the first receiving slot being sized to receive the distal end of the first pull wire and the second receiving slot being sized to receive the distal end of the second pull wire. The band may have a thickness, a first edge, a second edge, and a height between the first edge and second edge, the height being greater than the thickness. The first and second receiving slots each may extend within the band from the first edge to a distance from the second edge. For example, the distance from the second edge may be between approximately 10% to approximately 50% of the entire height of the band. The catheter steering assembly may further include a fusible thermoplastic band in contact with an inner surface of the band and a marker band located a predetermined distance from the first edge of the annular band.

A catheter steering assembly may include: a first pull wire having a distal end and a second pull wire having a distal end, the first and second pull wires each being flat in cross section; and a continuous annular band defining a first aperture, a second aperture, a first receiving slot, a second receiving slot, a thickness, a first edge, a second edge, and a height between the first edge and the second edge, the height being greater than the thickness, the first aperture being approximately 180° from the second aperture and the first receiving slot being approximately 180° from the second receiving slot, the first and second receiving slots each extending within the band from the first edge to a distance from the second edge, the distal end of the first pull wire being longitudinally received within the first receiving slot and the distal end of the second pull wire being longitudinally received within the second receiving slot.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows a system including a steering deflection mechanism;

FIG. 2 shows a cross-sectional view of a medical device elongate body including a steering deflection mechanism having a pull ring;

FIG. 3 shows a perspective view of a first embodiment of a pull ring;

FIG. 4 shows a top view of a round pull wire received within the first embodiment of the pull ring;

FIG. 5 shows a perspective view of a second embodiment of a pull ring; and

FIG. 6 shows a top view of a flat pull wire received within the second embodiment of the pull ring.

### DETAILED DESCRIPTION OF THE INVENTION

As used herein, the term “steering deflection anchor,” “deflection anchor,” or “pull wire anchor” refers to a pull ring to which one or more pull wires are attached. Further, the term “steering deflection mechanism” or “anchor mechanism” includes the pull ring (or deflection anchor) and pull wire, and may optionally include an inner layer of thermoplastic and a marker band.

As used herein, the term “round pull wire” refers to a longitudinally extended catheter pull wire that is round or circular in cross section. Likewise, the term “flat pull wire” refers to a longitudinally extended catheter pull wire that is flat or rectangular in cross section, at least in the distal portion of the pull wire.

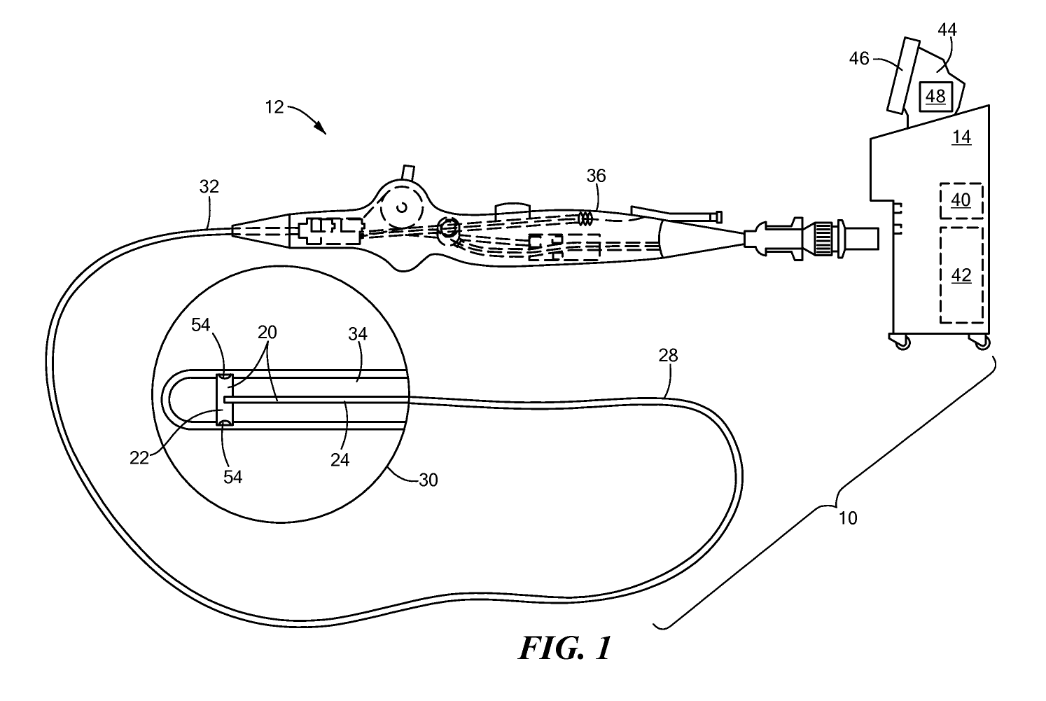

Referring now to FIG. 1, a system including a steering deflection mechanism is shown. The system 10 may generally include a medical device 12 (for example, an ablation, treatment, and/or mapping catheter) and a console 14. The medical device 12 may include a steering deflection mechanism 20, which may generally include a pull wire anchor 22 (also referred to herein as a pull ring) and one or more pull wires 24.

The medical device 12 may include an elongate body 28 having a distal portion 30, a proximal portion 32, and one or more lumens 34 therebetween. The distal portion 30 of the elongate body 28 may be capable of in-plane and/or out-of-plane deflection and is steerable by actuation of the one or more pull wires 24. The proximal portion 32 of the elongate body 28 may be affixed to a handle 36, which may have various inlets, outlets, steering control mechanisms (for example, knobs, toggles, etc.), and/or other components. Further, the one or more pull wires 24 may be either coupled to or routed through the handle 36.

The medical device 12 may be in fluid and/or electrical communication with the console 14. For example, the system 10 and device 12 may be configured for use with any of a variety of energy modalities (such as cryotreatment, radiofrequency energy, laser energy, pulsed field energy, microwave energy, ultrasound, energy or the like), for mapping tissue, and/or any other medical procedure that is facilitated by using a steerable catheter. As such, the console 14 may include one or more components appropriate for the purpose of the system. For example, the console 14 may include an energy generator 40 (for example, a radio frequency generator) and/or a refrigerant reservoir 42. The console 14 may further include a computer 44 having a display 46 and one or more processors 48 for receiving and processing data from the system 10, and/or various user control devices (for example, buttons, knobs, valves, keyboard, touch screen, foot pedals, etc.).

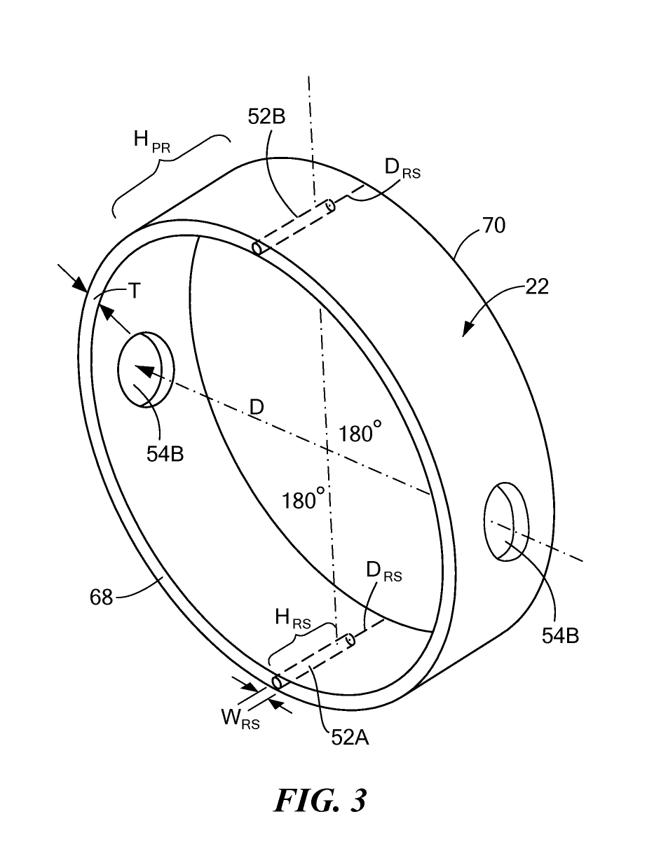

The pull wire anchor 22 (also referred to as “pull ring”) of the steering deflection mechanism 20 may be affixed to the inside of the distal portion 30 of the elongate body 28 and the one or more pull wires 24 may be coupled to the pull ring 22. When a user manipulates the pull wires 24, the distal portion 30 of the elongate body 28 may be deflected (that is, steered) in the direction of the force being exerted on the pull ring 22 by the one or more pull wires 24. As is shown in greater detail in FIGS. 3-6, the pull ring 22 may generally include one or more receiving slots 52, each of which receiving a pull wire 24, and a plurality of apertures 54.

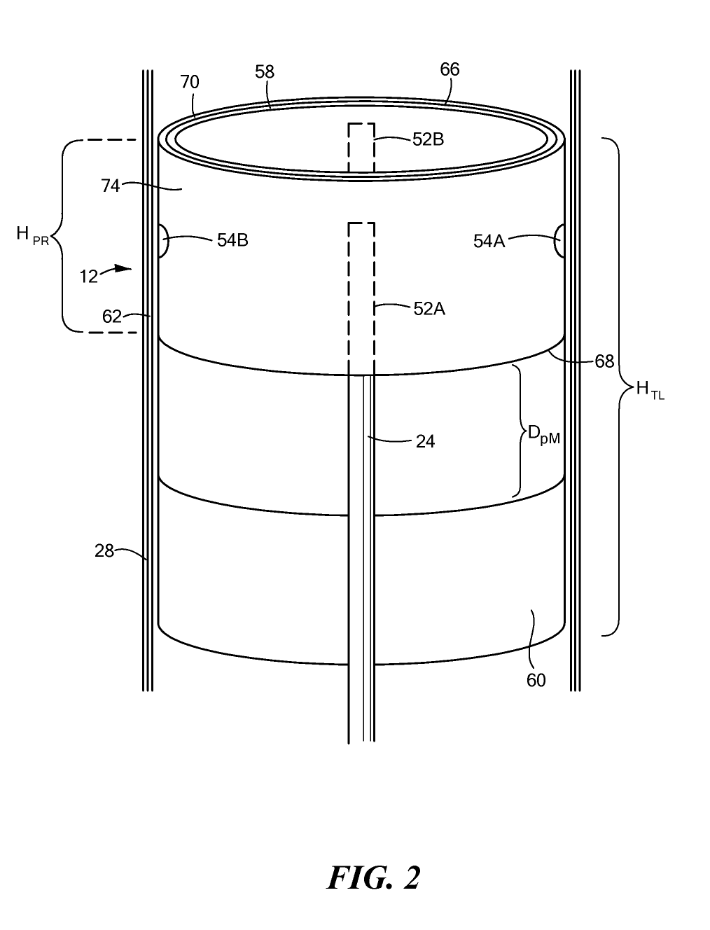

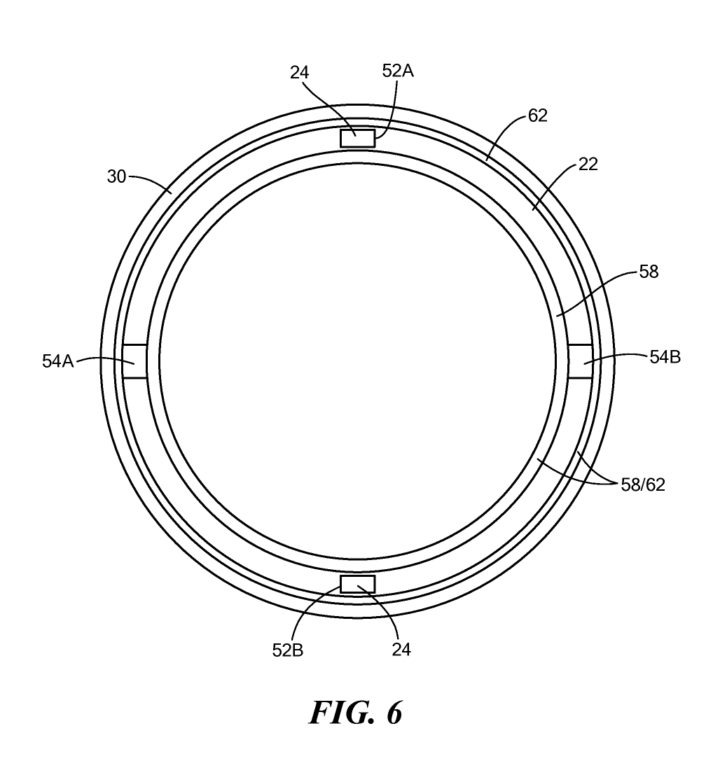

Referring now to FIG. 2, a cross-sectional view of a medical device elongate body including a steering deflection mechanism having a pull ring is shown. The medical device 12 may be a catheter, as shown in FIGS. 1 and 2. The steering deflection mechanism 20 (or anchor mechanism) as shown in FIG. 2 may generally includes a pull ring 22 and a pull wire 24 having a circular or round cross section (also referred to as a “round pull wire”). Alternatively, as described in FIGS. 3 and 6, the pull wire 24 may have a flat or rectangular cross section, at least in the distal portion of the pull wire 24. The pull ring 22 may have a height H_{PR}. The steering deflection mechanism 20 may further include an inner ring layer 58 of thermoplastic elastomer (for example, Pebax®) and a marker band 60, as shown in FIG. 2. The steering deflection mechanism 20 may be located in the distal portion 30 of the elongate body 28, and the elongate body 28 may include an inner lining 62 composed of the same thermoplastic as the inner ring layer 58. Thermoplastic in general, such as when the inner ring layer 58 and elongate body lining 62 melt together, is referred to as “thermoplastic 58/62.” The thermoplastic inner ring layer 58 may be in contact with the inner surface 66 of the pull ring 22. As a non-limiting example, the thermoplastic inner ring layer 58 may be approximately 0.05 mm to approximately 0.5 mm thick. Further, the thermoplastic inner ring layer 58 may have a height H_{TL} that is greater than the height H_{PR} of the pull ring 22. If a marker band 60 is included, the height H_{TL} of the thermoplastic inner ring layer 58 may be large enough to both line the inner surface 66 of the pull ring 22 and extend a substantial portion of a predetermined distance D_{PM} between the pull ring 22 and marker band 60. The distance D_{PM} between the pull ring 22 and marker band 60 may depend on such considerations as the procedure for which the device 12 will be used and the type of catheter navigation system used.

The steering deflection mechanism 20 may be coupled to the elongate body 28 by heating the elongate body 28 to a melting temperature of the thermoplastic 58/62 used in the steering deflection mechanism 20 and the elongate body inner lining 62. Specifically, as the thermoplastic 58/62 is heated and melts, the thermoplastic inner ring layer 58 of the steering deflection mechanism 20 may meld or blend with the thermoplastic inner lining 62 of the elongate body 28, thereby affixing the steering deflection mechanism 20 to the distal portion 30 of the device 12. Further, melted thermoplastic 58/62 may also flow through the apertures 54 in the pull ring 22 to give added strength to the point of connection between the anchor mechanism 20 and the elongate body 28 when the thermoplastic 58/62 hardens. Similarly, the marker band 60 may also become affixed to the distal portion 30 of the elongate body 28 by the thermoplastic 58/62. The round pull wire 24 may pass along the inside of the marker band 60 (as shown in FIG. 2) or on the outside of the marker band 60 (not shown).

The cross section shows a typical distribution of thermoplastic 58/62 between the elongate body inner lining 62 and the steering deflection mechanism 20. Melted thermoplastic 58/62 of the elongate body inner lining 62 and the inner ring layer 58 of the steering deflection mechanism 20 may meld or blend as the melting temperature of the thermoplastic 58/62 is reached. As the elongate body 28 is allowed to cool, the thermoplastic 58/62 may harden and thereby strengthen the point of connection between the distal portion 30 of the elongate body 28 and the steering deflection mechanism 20. Although not shown in the figures, the elongate body 28 may include additional layers or elements.

Referring now to FIG. 3, a perspective view of a first pull ring embodiment is shown. The pull ring 22 may generally include a first receiving slot 52A, a second receiving slot 52B, and a plurality of apertures 54. The pull ring 22 is an annular band having a height H_{PR}, a thickness T, and a diameter D. The height H_{PR} is greater than the thickness T, and the thickness T may be greater than commonly known pull rings in order to accommodate a portion of each of the pull wires 24 therein. The pull ring 22 may have any dimensions that are appropriate for the pull wire 24 and device 12 assembly used. The pull ring 22 may also have a first edge 68 and a second edge 70, used herein as reference points for describing the locations of other features of the system and method. Further, the pull ring 38 may be composed of any suitable material, such as stainless steel, titanium, Nitinol, or alloy such as 304V spring temper.

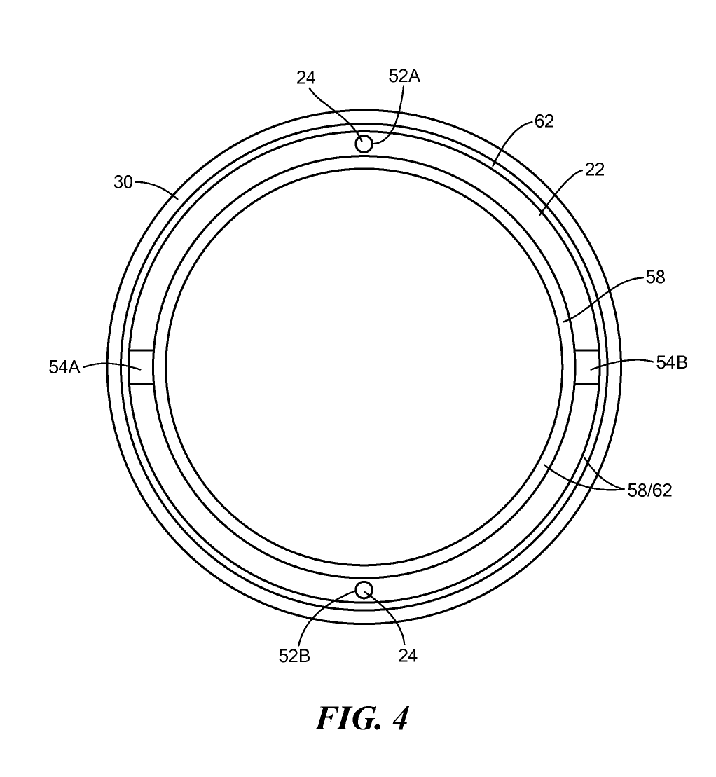

Continuing to refer to FIG. 3, the receiving slots 52A, 52B may have a circular cross section for receiving a straight rounded pull wire 24 (as shown in FIG. 4). The receiving slots 52A, 52B of FIG. 3 each may be entirely contained within the pull ring 22, such that the portion of pull wire 24 disposed within each receiving slot may be concealed by the pull ring 22. That is, the width W_{RS} of each receiving slot 52A, 52B may be less than the thickness T of the pull ring 22. Each receiving slot 52A, 52B may have an opening in the first edge 68 of the pull ring 22 and continue from the first edge 68 within the pull ring 22 to a distance D_{RS} from the second edge 70. Thus, the second edge 70 may be at least substantially continuous without any receiving slot apertures. As a non-limiting example, the width W_{RS} of the receiving slots 52A, 52B may be approximately 0.30 mm (or any width that is slightly larger than the diameter of the pull wire 24 and can receive the pull wire without too much gapping between the wire and the inner walls of the receiving slot), and the height H_{RS} may be approximately 1.12 mm. The distance D_{RS} between the receiving slots 52A, 52B and the second edge 70 of the pull ring 22 may be, for example, between approximately 15% and approximately 55% of the height of the height H_{PR} of the pull ring 22. However, the receiving slots 52A, 52B and pull ring 22 may have any measurements that are appropriate for the pull wire 24 and device 12 assembly used.

Continuing to refer to FIG. 3, the pull ring 22 may include any number of apertures 54, for example, two or more. The apertures 54 may allow melted Pebax or similar thermoplastic 58/62 to flow through, each aperture 54 thereby serving as an anchor point between the elongate body inner lining 62 and the pull ring 22 (as shown and described in FIG. 6). Accordingly, the apertures 54 may be distributed about the pull ring 22 in a pattern that will counteract destructive pull forces exerted on the pull wire 24. For example, the pull ring 22 may have a first aperture 54A and a second aperture 54B, each aperture 54A, 54B being located equidistant from the first 68 and second edge 70 of the pull ring 22. The first aperture 54A may be located approximately 180° from the second aperture 54B, and the first receiving slot 52A may be located approximately 180° from the second receiving slot 52B. That is, the first 54A and second 54B apertures may be diametrically opposed from each other and the first 52A and second 52B receiving slots may be diametrically opposed from each other. Further, the first aperture 54A may be approximately 90° from each of the first 52A and second 52B receiving slots. The apertures 54A, 54B may each have approximately the same diameter or they may be of different sizes and shapes.

Referring now to FIG. 4, a top view of a round pull wire received within the first embodiment of the pull ring 22 is shown. The inner surface 66 and the outer surface 74 of the pull ring 22 may have the same or different textures, such as smooth, ridged, knurled, scored, or the like. Because the round pull wire 24 has a diameter slightly smaller than the width W_{RS} of the receiving slots 52A, 52B, the round pull wires 24 may be received within the receiving slots 52A, 52B and affixed to the pull ring 22. For example, each pull wire 24 may be inserted into the receiving slots 52A, 52B and the pull wires 24 may be laser welded to the outer surface of the pull ring 22, such as at the location where the exposed portion of each pull wire 24 is immediately adjacent to the opening of its receiving slot 52A, 52B. Optionally, an adhesive or chemical bonding material, for example, may be used within the receiving slots 52A, 52B to strengthen the coupling between the pull wires 24 and the pull ring 22. Additionally or alternatively, other bonding, adhesion, or coupling means may be used, such as friction fitting, adhesives, or chemical bonding. For example, the pull wires 24 may be attached to the pull ring 22 using a thermoplastic, by laser welding, and/or by a cyanoacrylate bond. The configuration of the receiving slots 52A, 52B and apertures 54A, 54B may allow for dual steering of the distal portion 30 of the elongate body 28, thereby allowing the operator to navigate the device 12 to a target treatment location more easily.

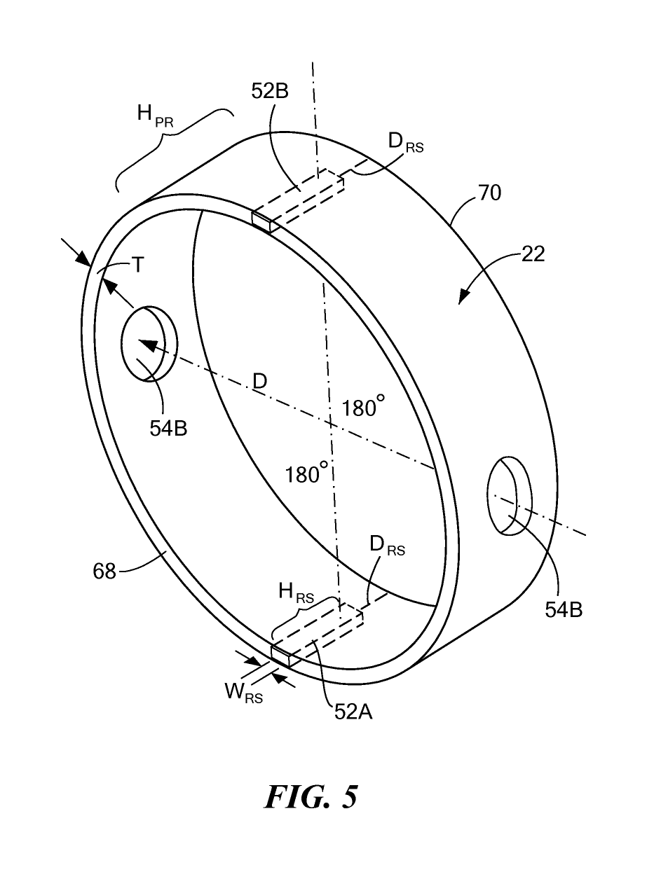

Referring now to FIGS. 5 and 6, a perspective view and a top view of a second embodiment of a pull ring are shown. The pull ring 22 of FIGS. 5 and 6 and manner of attaching the pull wires 24 thereto is similar to the pull ring 22 of FIGS. 3 and 4, except for the cross section of the receiving slots 52A, 52B. As is shown in FIGS. 5 and 6, the receiving slots 52A, 52B of the pull ring 22 may have a rectangular cross section to accommodate a flat pull wire 24 or round pull wire with a flat distal portion. The wider legs 76 of the rectangular cross section may be substantially in line with the pull ring 22, whereas the narrower legs 78 of the rectangular cross section may extend between the pull ring inner surface 66 and the pull ring outer surface 74. The height H_{RS} of the rectangular receiving slots 52A, 52B may be similar to that of the round receiving slots 52A, 52B of FIG. 3.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A catheter steering assembly, the assembly comprising:
a first pull wire and a second pull wire; and
a continuous annular band including therein a first aperture, a second aperture, and a first receiving slot, and a second receiving slot, the first aperture being approximately 180° from the second aperture and the first receiving slot being approximately 180° from the second receiving slot,
at least a portion of the first pull wire being received within the first aperture and at least a portion of the second pull wire being received within the second aperture.

2. The assembly of claim 1, wherein the first and second pull wires each have a circular cross section.

3. The assembly of claim 2, wherein each of the first and second receiving slots have a circular cross section that is sized to longitudinally receive a portion of one of the first pull wire and the second pull wire.

4. The assembly of claim 1, wherein the first and second pull wires each have a rectangular cross section.

5. The assembly of claim 4, wherein each of the first and second receiving slots have a rectangular cross section that is sized to longitudinally receive a portion of one of the first pull wire and the second pull wire.

6. The assembly of claim 1, wherein the band includes a thickness, a first edge, a second edge, and a height between the first edge and second edge, the height being greater than the thickness.

7. The assembly of claim 1, wherein the first and second apertures are each circular in shape.

8. The assembly of claim 1, wherein the first and second apertures are each equidistant from the first edge and second edge.

9. The assembly of claim 8, wherein the first and second receiving slots each extend within the band from the first edge to a distance from the second edge.

10. The assembly of claim 9, wherein the distance from the second edge is between approximately 10% to approximately 50% of the entire height of the band.

11. The assembly of claim 1, further comprising a fusible band in contact with an inner surface of the band.

12. The assembly of claim 11, wherein the fusible band is composed of a thermoplastic.

13. A catheter steering assembly, the assembly comprising:
a first pull wire having a distal end and a second pull wire having a distal end, the first and second pull wires each being flat in cross section; and
a continuous annular band including therein a first aperture, a second aperture, a first receiving slot and a second receiving slot, the first aperture being approximately 180° from the second aperture and the first receiving slot being approximately 180° from the second receiving slot, the first receiving slot being sized to receive the distal end of the first pull wire and the second receiving slot being sized to receive the distal end of the second pull wire.

14. The assembly of claim 13, wherein the band includes a thickness, a first edge, a second edge, and a height between the first edge and second edge, the height being greater than the thickness.

15. The assembly of claim 13, wherein the first and second apertures are each circular in shape.

16. The assembly of claim 15, wherein the first and second receiving slots each extend within the band from the first edge to a distance from the second edge.

17. The assembly of claim 16, wherein the distance from the second edge is between approximately 10% to approximately 50% of the entire height of the band.

18. The assembly of claim 13, further comprising a fusible thermoplastic band in contact with an inner surface of the band.

19. The assembly of claim 18, further comprising a marker band located a predetermined distance from the first edge of the annular band.

20. A catheter steering assembly, the assembly comprising:
a first pull wire having a distal end and a second pull wire having a distal end, the first and second pull wires each being flat in cross section; and
a continuous annular band defining a first aperture, a second aperture, a first receiving slot, a second receiving slot, a thickness, a first edge, a second edge, and a height between the first edge and the second edge, the height being greater than the thickness,
the first aperture being approximately 180° from the second aperture and the first receiving slot being approximately 180° from the second receiving slot, the first and second receiving slots each extending within the band from the first edge to a distance from the second edge, the distal end of the first pull wire being longitudinally received within the first receiving slot and the distal end of the second pull wire being longitudinally received within the second receiving slot.
